(19) United States
(12) Patent Application Publication
(10) Pub. No.: US 2023/0346537 A1
(43) Pub. Date: Nov. 2, 2023

(54) Graft Device for Endogenous Tissue Restoration in Between Two Tubular Structures

(71) Applicant: XELTIS AG, Zurich (CH)

(72) Inventors: Yonatan Gray, Paris (FR); El-Kurdi Mohammed, Mansfield, MA (US)

(21) Appl. No.: 18/025,955

(22) PCT Filed: Nov. 12, 2021
(86) PCT No.: PCT/EP2021/081445

§ 371 Date: Mar. 13, 2023

Related U.S. Application Data
(60) Provisional application No. 63/113,283, filed on Nov. 13, 2020.

(51) Int. Cl. A61F 2/07; A61F 2/915; A61F 2/00

(52) U.S. Cl. CPC A61F 2/07; A61F 2/915; A61F 2/0077; A61F 2002/0086; A61F 2210/0004; A61F 2210/0076; A61F 2240/001; A61F 2002/072

(57) ABSTRACT

Graft devices are provided addressing a long need for off-the-shelf small diameter replacement vessels to overcome the drawbacks of currently available alternatives. As they are available off-the-shelf, the graft devices do not require additional surgery to harvest it such as for a vein graft. A porous nature of the graft devices enables restoration process, which results in new natural and patient-own tissue, in contrast to currently existing vascular prosthesis that can never fully heal. A built-in graft support device over-comes the limited kink-resistance that is typical for these kinds of (electro-spun) porous devices. A zigzag pattern with alternating laminating and non-laminating areas enables the incorporation of the graft support device without the need for additional suturing or connecting the inner and outer layer for good lamination, while maintaining adequate kinkresistance.

### FIELD OF THE INVENTION

This invention relates to graft devices and methods capable of promoting endogenous tissue restoration or growth.

### BACKGROUND OF THE INVENTION

There is long remaining need for off-the-shelf small diameter replacement vessels to overcome the drawbacks of currently available alternatives.

Coronary Artery Bypass Grafting (CABG) is the most common open heart surgical procedure and is performed more than a million times per year worldwide. In 80% of CABG procedures, native vein segments (2-3 on average per procedure) are used for coronary revascularization, thus requiring an additional painful surgical procedure to harvest the vein from the patient's leg, which is often associated with complications such as infections and chronic pain. Despite rigorous efforts, no synthetic off-the-shelf alternatives for this application exist today. Commonly used vascular prosthesis, such as those based on ePTFE or Dacron are not commercially available for CABG as these grafts fail to remain patent (open) in diameters of 4 mm and below as required for CABG.

Furthermore, in other small diameter vessel applications, such as dialysis access grafts or in peripheral applications such as Critical Limb Ischemia (CLI) there is still a large unmet medical need, despite the slightly larger diameters (typically 6, and up to 8 mm), both native veins and prosthetic grafts do not provide satisfactory long-term patency. The problem with prosthetic grafts in these applications is that they do not allow restoration of natural tissue, and therefore they cannot heal adequately. Eventually, these grafts occlude because deposited proteins and tissues from the blood stream accumulate within these grafts with time, eventually resulting in stenosis and occlusion.

Accordingly, there is a need in the art to provide a graft device for restoring a vessel by being capable of promoting endogenous tissue restoration or growth, while maintaining the structural and dynamical requirements desired for a graft device. The present invention provides a graft device that addresses this need.

### SUMMARY OF THE INVENTION

### Definitions

For the purposes of this invention, the term graft is defined as grafts that are used to create a connection between 2 blood vessels, which could be a bypass graft, a shunt, an interposition graft, end-to-end, side-to-end, end-to-side, side-to-side, including snake and jump grafts (where several bypasses are made with one graft). What is not meant is devices that are used inside an existing blood vessel such as stents, endografts etc.
Small diameter ranges of graft devices provided herein are defined as 4 mm or less (for CABG), around 6 mm (for access graft) and up to 8 mm for peripheral grafts.

The present invention provides a graft device for endogenous tissue restoration in between two tubular structures. In one embodiment, the graft device distinguishes an electrospun inner tubular layer, an electrospun outer tubular layer; and a graft support device defined as a zig-zag patterned helix having an inner tubular surface and an outer tubular surface. The electrospun inner tubular layer matches the inner tubular surface, and the electrospun outer tubular layer matches the outer tubular surface. Together the electrospun inner tubular layer and the electrospun outer tubular layer sandwich the graft support device. In one embodiment, the zig-zag patterned helix takes up about 95% of the length of the graft device.

The graft device is deployable in a predetermined state or wherein the graft device maintains a predetermined state upon implantation.

The graft support device further distinguishes first areas defined by the corners of the zig-zag pattern, and second areas defined by areas within each V or inverted-V within the zig-zag pattern minus the first area defined as their respective corners.

The first areas are non-laminated areas where the electrospun inner tubular layer and the electrospun outer tubular layer are not-laminated together. These first non-laminated areas enable bending of the graft support device, while preventing kinking of the graft support device. In one example, the first non-laminated area for each corner has a surface area in a range of 0.3 to 0.5 mm².

The second areas are laminated areas where the electrospun inner tubular layer and the electrospun outer tubular layer are laminated together. In one example, the second laminated area for each within each V or inverted-V has a surface area in a range of 2.5 to 3.5 mm².

In one embodiment, the graft support device is made out of a metal or a polymer, and the electrospun inner and outer tubular layer are made out of polymer fibers, and where the second areas have a polymer to helix metal or helix polymer circumferential surface area ratio ranging from 4:1 to 12:1 (8:1).

In yet a further embodiment, each corner within the graft support device is an n-like shape or a u-like shape depending on the direction within the zig-zag pattern and each corner has a surface area in a range of 0.3 to 0.5 mm². The graft support device has a uniform pitch angle.

In yet a further embodiment, the electrospun inner and outer tubular layer are each porous biodegradable polymer layers with a porosity large enough to allow for cell ingrowth upon implantation to promote the endogenous tissue restoration or growth. The electrospun inner and outer tubular layer are replaced over time by the endogenous tissue restoration or growth as a result of the cell ingrowth.

In yet a further embodiment, the graft support device at one end or at both ends has one or more independent C-rings distributed and positioned at an acute orientation angle relative to a longitudinal axis of the graft device.

In yet a further embodiment, the graft support device at one end or at both ends have a closed ring connected to the graft support device.

In still another embodiment, the invention provides a graft device distinguishing an electrospun inner tubular layer, an electrospun outer tubular layer, and a graft support device defined as a patterned helix having an inner tubular surface and an outer tubular surface. Similarly, as the graft device described above, the electrospun inner tubular layer matches the inner tubular surface, the electrospun outer tubular layer matches the outer tubular surface, and together the electrospun inner tubular layer and the electrospun outer tubular layer sandwich the patterned helix distinguishing laminated areas and non-laminated areas. The non-laminated areas enable bending of the patterned helix, while preventing kinking of the graft support device.

In still another embodiment, the invention provides a method of creating a connection between two tubular structures using a graft device. Here the graft device distinguishes an electrospun inner tubular layer, an electrospun outer tubular layer, and a graft support device defined as a patterned helix having an inner tubular surface and an outer tubular surface. Similarly, as the graft devices described above, the electrospun inner tubular layer matches the inner tubular surface, the electrospun outer tubular layer matches the outer tubular surface, and together the electrospun inner tubular layer and the electrospun outer tubular layer sandwich the patterned helix distinguishing laminated areas and non-laminated areas. The non-laminated areas enable bending of the patterned helix, while preventing kinking of the graft support device. After implantation of the graft device, the electrospun inner and outer tubular layer are substantially replaced over time by the endogenous tissue restoration or growth as a result of the cell ingrowth.

Embodiments of this invention have the following advantages:
They are available off-the-shelf and do not require additional surgery to harvest it such as for a vein graft.
The porous nature of the graft devices enables restoration process, which results in new natural and patient-own tissue, in contrast to currently existing vascular prosthesis that can never fully heal.
The built-in graft support device overcomes the limited kink-resistance that is typical for these kinds of (electrospun) porous devices.
The zig-zag pattern with the alternating laminating and non-laminating areas enables the incorporation of the graft support device without the need for additional suturing or connecting the inner and outer layer for good lamination, while maintaining adequate kink-resistance.

### BRIEF DESCRIPTION OF THE DRAWINGS

FIG. 1 shows according to an exemplary embodiment of the invention a cross-section of the graft support device.

FIG. 2 shows according to an exemplary embodiment of the invention a portion of the zig-zag helix pattern of the graft support device in a side view. Also shown is the definition of the pitch angle in relation to the side view.

FIG. 3 shows according to an exemplary embodiment of the invention a portion of the zig-zag patterned helix of the graft support device in a side view indicating the first areas (circles) which won't get laminated and the second areas (triangles) which will get laminated once the zig-zag patterned helix is sandwiched in between the inner and outer electrospun tubular layers.

FIG. 4 shows according to an exemplary embodiment of the invention a portion zig-zag helix pattern of the graft support device with bridges between revolutions of the helix.

FIG. 5 shows according to an exemplary embodiment of the invention an actual design of the graft support device showing the first areas which are the corner areas (i.e. the n and u corner shapes as shown in FIG. 3).

FIG. 6 shows according to an exemplary embodiment of the invention a graft support device with a patterned helix with on one or both ends C-rings.

### DETAILED DESCRIPTION

There is a need in the art to provide a graft device for restoring a vessel by being capable of promoting endogenous tissue restoration or growth, while maintaining the structural and dynamical requirements desired for a graft device. The present invention provides a graft device that addresses this need.

In one embodiment, the graft device is a tubular implant for making an anastomotic connection in between two tubular structures. Examples of tubular implants include, without limitation, a vein, an artery, a urethra, an intestine, an esophagus, a trachea, a bronchii, a ureter, or a fallopian tube. The graft device intended in this invention is not a device for endoluminal placement—i.e. inside the lumen of an existing tubular structure.

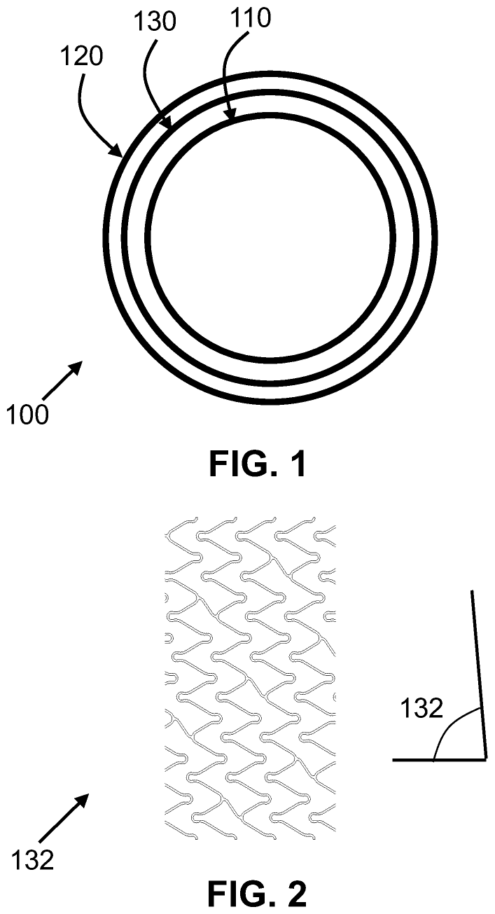

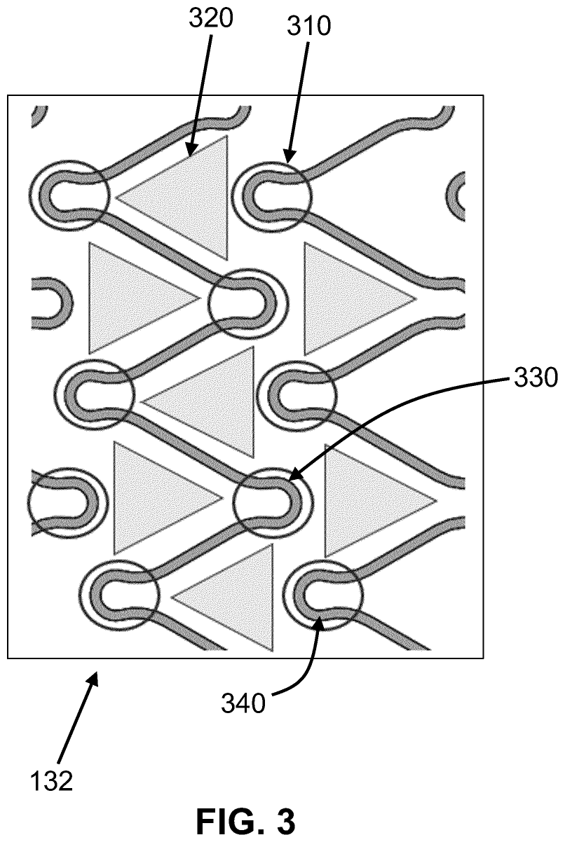

In one embodiment, the graft device 100 has an electrospun inner tubular layer 110 and an electrospun outer tubular layer 120 (FIG. 1) A graft support device formed from a zig-zag patterned helix 130, for which an exemplary section is shown in FIG. 2, is sandwiched in between the electrospun inner tubular layer 110 and the electrospun outer tubular layer 120. The zig-zag patterned helix 130 has a uniform pitch angle 132 (FIG. 2), an inner tubular surface, and an outer tubular surface. The electrospun inner tubular layer 110 matches the inner tubular surface, and the electrospun outer tubular layer 120 matches the outer tubular surface. The inner tubular layer 110 is in contact with the outer tubular layer 120, except where the zig-zag patterned helix 130 is in between.

Embodiments of this invention are not limited to a graft support device from a zig-zag patterned helix as long as the patterned helix can achieve the goal of a graft device with laminated and non-laminated areas with the objectives for bending enablement and kinking prevention. The device has an electrospun inner and outer tubular layer with a patterned helix having an inner tubular surface and an outer tubular surface. The electrospun inner tubular layer matches the inner tubular surface, and the electrospun outer tubular layer matches the outer tubular surface. Together the electrospun inner tubular layer and the electrospun outer tubular layer sandwich the patterned helix distinguishing laminated areas and non-laminated areas. The non-laminated areas enable bending of the patterned helix, while preventing kinking of the zig-zag patterned helix.

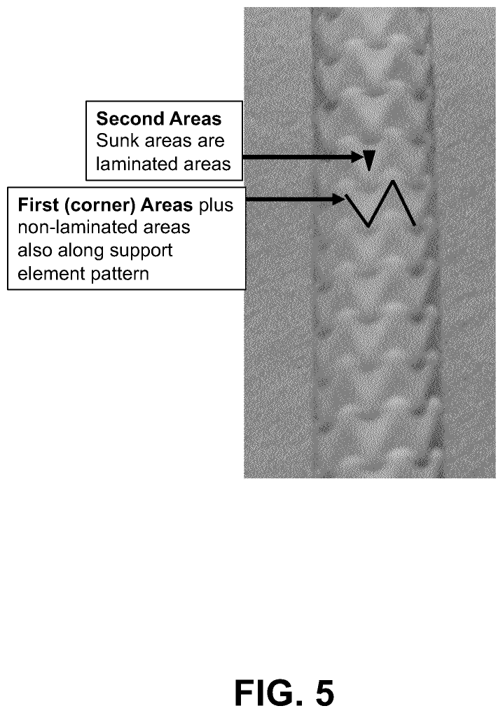

Referring back to the example of the zig-zag patterned helix, this pattern distinguishes first areas 310 defined by the corners of the zig-zag pattern (FIGS. 3 and 5). The first areas are non-laminated areas where the electrospun inner tubular layer and the electrospun outer tubular layer are not-laminated together due to a relatively high density of material of the corners of the zig-zag patterns. The electrospun material cannot connect to each other in tight spaces like first areas 310, which enhances kink resistance while also enabling bending of the zig-zag patterned helix. In one example, the first non-laminated areas for each corner each have a surface area in a range of 0.3 to 0.5 mm².

The zig-zag patterned helix distinguishes second areas 320 defined by areas within each V or inverted-V within the zig-zag pattern minus the first area defined as their respective corners (FIGS. 3 and 5). The second areas are laminated areas where the electrospun inner tubular layer and the electrospun outer tubular layer are laminated together and stay laminated or adhered together. In one example, the second laminated areas within each V or inverted-V have a surface area in a range of 2.5 to 3.5 mm².

The zig-zag patterned helix can be made out of a metal (e.g. nitinol) or a polymer, and the electrospun inner and outer tubular layer can be made out of polymer fibers. In one embodiment, electrospun polymer to metal (or polymer) circumferential/cylindrical surface area ratio ranges from 4:1 to 12:1 (defined for the graft device). In one exemplary embodiment this ratio is about 8:1. The circumferential/cylindrical surface area is measured on the outer surface of the graft supporting device.

It is important for the embodiments that the electrospun inner and outer tubular layer are each porous biodegradable polymer layers with a porosity large enough to allow for cell ingrowth upon implantation to promote the endogenous tissue restoration or growth. The electrospun inner and outer tubular layers are replaced over time by the endogenous tissue restoration or growth as a result of the cell (in)growth.

For the specific design of the graft support device in this invention each corner within the zig-zag patterned helix is an n-like shape 330 or a u-like shape 340 depending on the direction within the zig-zag pattern as shown in FIG. 3. This in contrast to v-like or inverted v-like corners, which would be much more prone to polymer damage by metal abrasion and does not maximize the desired first non-laminated areas needed for zig-zag patterned helix mobility.

The n-like shape or the u-like shape are narrow and as such do not allow electrospun inner and outer polymer fibers to adhere/bond locally to each other, i.e. remain delaminated. Rather these n-like shape or a u-like shapes serve as “hinge areas” where relative movement between the helix and the electrospun layers is possible due to relative high metal density and where local electrospun polymer fibers are not able to interconnect through the u-like or n-like shaped structures of the metal/polymer (i.e. first areas).

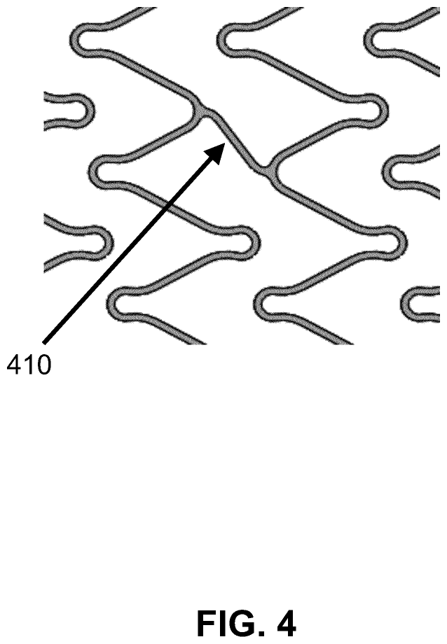

The zig-zag patterned helix can be made out of a laser cut tube. In some embodiments, connecting struts (“bridges”) 410 can be considered to improve manufacturing yield (FIG. 4). Connectors can be designed in a way to not compromise on structure's ability to recover from severe clamping nor to decrease its fatigue life endurance. As such, the number of bridge connectors can vary from none to a plurality of bridges per revolution. The bridge configuration can be used to tune the axial compliance of the graft device.

In one example, the uniform pitch angle 132 as shown in FIG. 2, defined between two adjacent revolutions of the (metal) support of the zig-zag helix is spaced in a way to allow strong polymer fiber attachment between the inner and outer electrospun layers. The pitch is around 2 mm. It should not be too high to prevent kinking. If this value is too small it will collapse. Preferred values are 1.5-2.5 mm but 1 to 3 mm should work as well.

The uniform pitch angle 132 as shown in FIG. 2 is roughly the same along the length of the zig-zag helix pattern. Preferred ratio of cell-to-cell distance to pitch is 1:1. A ratio of 2:1.5 up to 1:1.5 can work as well, a ratio lower than 1:1.5 or alternatively a ratio higher than 2:1 will result in compromised kink resistance. The favorable distance between two adjacent cells was found to be 2 mm, hence—pitch is optimally set as well to 2 mm. This leads to an optimal opening and provides the support structure with excellent kink-resistance.

The graft manufacturing process starts by electrospinning of an inner layer on tubular mandrel. The inner layer is spun such that its outer diameter corresponds to the inner diameter of the graft support device, resulting in sufficient friction between the two components. The graft support device is then expanded and loaded on a tube. The tube inner diameter is larger than the spun inner layer outer diameter such that it serves as a deployment tool for the graft support device to be deployed in its desired location axially over the inner layer. The next step is to electrospin the outer layer, in a special process designed to reach optimal adherence (i.e. lamination) of the outer layer fibers to those of the inner layer at the non-metal covered areas. This process assures that the second areas are fully laminated and was tested and validated on benchtop. The aforementioned specifications of the support element (i.e. polymer to support element density, cell to cell spacing) were designed to result in optimal lamination of the fibers.

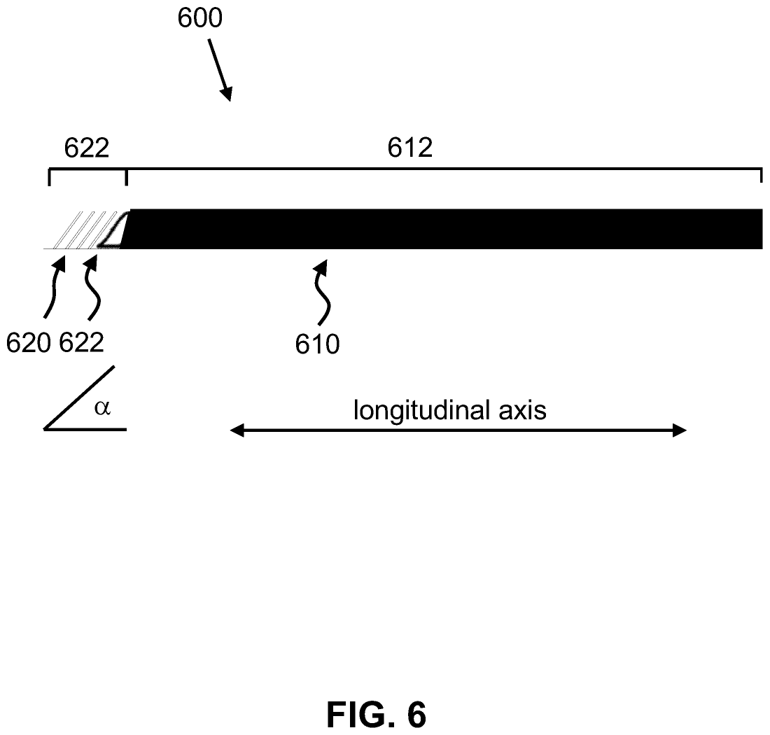

In a further embodiment shown in FIG. 6, a graft support device 600 defines a longitudinal axis. The main body of the graft support device is made of the patterned helix as for example shown in FIGS. 2-3. In one embodiment 90-95% of the length 612 of the graft support device defined in direction of the longitudinal axis is that patterned helix. For the other about 5-10% 622, one or more independent C-rings 620 are distributed and positioned at an acute orientation angle α relative to the longitudinal axis of the graft device at one end of the support element, and potentially also at the other end of the graft support device (not shown). The C-rings are embedded in between the electrospun inner and outer tubular layers. Depending on the application the of acute orientation angle could be a 15-90 degree-angle or preferably a 30-60 degree angle, or nominally a 45 degree angle.

C-rings are defined as either a circular or oval ring that is not fully closed; i.e. has an opening, large enough to accommodate standard surgical scissors for axial slit creation without cutting through the ring strut. In one embodiment, the openings of the C-rings are aligned with each other. In an alternate embodiment, the C-rings could be closed rings.

The C-rings are embedded in between the inner and outer tubular layers, in a way that prevents delamination of the layers. In one embodiment, the orientation angle is nominally about 45 degrees. In a preferred embodiment, the C-rings are made of nitinol.

In one embodiment, the patterned helix part of the graft support device 612 has an oval or circular end-ring 624 attached to (and part of) the patterned helix part. This so-called end-ring 624 is aligned more or less in parallel to the two or more independent C-rings. In a preferred embodiment, the end ring is made of nitinol.

Note is that this end-ring is physically connected to the graft support device. This ring is always fully closed. This is important as it prevents the graft from collapsing and stabilizes the end part of the graft. Furthermore, it makes the graft support device non-expandable and different from endoluminal devices such as stents.

The electrospun material referenced in this document may comprise the ureido-pyrimidinone (UPy) quadruple hydrogen-bonding motif (pioneered by Sijbesma (1997), Science 278, 1601-1604) and a polymer backbone, for example selected from the group of biodegradable polyesters, polyurethanes, polycarbonates, poly(orthoesters), polyphosphoesters, polyanhydrides, polyphosphazenes, polyhydroxyalkanoates, polyvinylalcohol, polypropylenefumarate. Examples of polyesters are polycaprolactone, poly(L-lactide), poly(DL-lactide), poly(valerolactone), polyglycolide, polydioxanone, and their copolyesters. Examples of polycarbonates are poly(trimethylenecarbonate), poly(dimethyltrimethylenecarbonate), poly(hexamethylene carbonate).

The same result may be obtained with alternative, non-supramolecular polymers, if properties are carefully selected and material processed to ensure required surface characteristics. These polymers may comprise biodegradable or non-biodegradable polyesters, polyurethanes, polycarbonates, poly(orthoesters), polyphosphoesters, polyanhydrides, polyphosphazenes, polyhydroxyalkanoates, polyvinylalcohol, polypropylenefumarate. Examples of polyesters are polycaprolactone, poly(L-lactide), poly(DL-lactide), poly(valerolactone), polyglycolide, polydioxanone, and their copolyesters. Examples of polycarbonates are poly(trimethylenecarbonate), poly(dimethyltrimethylenecarbonate), poly(hexamethylene carbonate).

What is claimed is:

1. A graft device for endogenous tissue restoration in between two tubular structures, comprising:
(a) an electrospun inner tubular layer;
(b) an electrospun outer tubular layer; and
(c) a graft support device defined as a zig-zag patterned helix having an inner tubular surface and an outer tubular surface,
wherein the electrospun inner tubular layer matches the inner tubular surface,
wherein the electrospun outer tubular layer matches the outer tubular surface,
wherein together the electrospun inner tubular layer and the electrospun outer tubular layer sandwich the graft support device,
wherein the graft support device distinguishes first areas defined by the corners of the zig-zag pattern,
wherein the graft support device distinguishes second areas defined by areas within each V or inverted-V within the zig-zag pattern minus the first area defined as their respective corners,
wherein the first areas are non-laminated areas where the electrospun inner tubular layer and the electrospun outer tubular layer are not-laminated together,
wherein the first non-laminated areas enable bending of the graft support device, while preventing kinking of the graft support device, and
wherein the second areas are laminated areas where the electrospun inner tubular layer and the electrospun outer tubular layer are laminated together.

2. The graft device as set forth in claim 1, wherein the graft support device is made out of a metal or a polymer, wherein the electrospun inner and outer tubular layer are made out of polymer fibers, and wherein the second areas have a polymer to helix metal or helix polymer circumferential surface area ratio ranging from 4:1 to 12:1 (8:1).

3. The graft device as set forth in claim 1, wherein the first non-laminated area for each corner has a surface area in a range of 0.3 to 0.5 mm².

4. The graft device as set forth in claim 1, wherein the second laminated area for each within each V or inverted-V has a surface area in a range of 2.5 to 3.5 mm².

5. The graft device as set forth in claim 1, wherein the electrospun inner and outer tubular layer are each porous biodegradable polymer layers with a porosity large enough to allow for cell ingrowth upon implantation to promote the endogenous tissue restoration or growth.

6. The graft device as set forth in claim 5, wherein the electrospun inner and outer tubular layer are replaced over time by the endogenous tissue restoration or growth as a result of the cell ingrowth.

7. The graft device as set forth in claim 1, wherein each corner within the graft support device is an n-like shape or a u-like shape depending on the direction within the zig-zag pattern and each corner has a surface area in a range of 0.3 to 0.5 mm².

8. The graft device as set forth in claim 1, wherein the graft support device has a uniform pitch angle.

9. The graft device as set forth in claim 1, wherein the graft support device at one end or at both ends has one or more independent C-rings distributed and positioned at an acute orientation angle relative to a longitudinal axis of the graft device.

10. The graft device as set forth in claim 1, wherein the graft support device at one end or at both ends have a closed ring connected to the graft support device.

11. The graft device as set forth in claim 9, wherein the zig-zag patterned helix takes up about 95% of the length of the graft device.

12. The graft device as set forth in claim 1, wherein the graft device is deployable in a predetermined state or wherein the graft device maintains a predetermined state upon implantation.

13. A graft device, comprising:
(a) an electrospun inner tubular layer;
(b) an electrospun outer tubular layer; and
(c) a graft support device defined as a patterned helix having an inner tubular surface and an outer tubular surface,
wherein the electrospun inner tubular layer matches the inner tubular surface,
wherein the electrospun outer tubular layer matches the outer tubular surface, and
wherein together the electrospun inner tubular layer and the electrospun outer tubular layer sandwich the patterned helix distinguishing laminated areas and non-laminated areas, wherein the non-laminated areas enable bending of the patterned helix, while preventing kinking of the graft support device.

14. A method of creating a connection between two tubular structures using a graft device, wherein the graft device comprises:
(a) an electrospun inner tubular layer;
(b) an electrospun outer tubular layer; and
(c) a graft support device defined as a patterned helix having an inner tubular surface and an outer tubular surface,
wherein the electrospun inner tubular layer matches the inner tubular surface,
wherein the electrospun outer tubular layer matches the outer tubular surface,
wherein together the electrospun inner tubular layer and the electrospun outer tubular layer sandwich the patterned helix distinguishing laminated areas and non-laminated areas, wherein the non-laminated areas enable bending of the patterned helix, while preventing kinking of the graft support device, and
wherein the electrospun inner and outer tubular layer after implantation are substantially replaced over time by the endogenous tissue restoration or growth as a result of the cell ingrowth.
